(19) United States
(12) Patent Application Publication
(10) Pub. No.: US 2004/0122385 A1
(43) Pub. Date: Jun. 24, 2004

(54) Absorbent articles including an odor absorbing and/or odor reducing additive

(75) Inventors: Michael Tod Morman, Alpharetta, GA (US); Roger Bradshaw Quincy III, Cumming, GA (US)

Correspondence Address: KIMBERLY-CLARK WORLDWIDE, INC., 401 NORTH LAKE STREET, NEENAH, WI, 54956

(73) Assignee: Kimberly-Clark Worldwide, Inc.

(21) Appl. No.: 10/327,822

(22) Filed: Dec. 23, 2002

(51) Int. Cl. A61F 13/15; A61F 13/20

(52) U.S. Cl. 604/359

(57) ABSTRACT

An absorbent article comprising a capturing agent and a liquid, wherein the liquid is captured by the capturing agent, for example disposed within or otherwise encapsulated within an encapsualting agent or complexed with a complexing agent is provided.

### FIELD OF THE INVENTION

The present invention relates to absorbent articles that include odor reducing additives.

### BACKGROUND OF THE INVENTION

Body fluids such as urine, menses, feces and so forth contain many types of odorous compounds. A variety of absorbent personal care articles such as diapers, adult incontinence products, feminine hygiene products and so forth are designed to absorb body fluids but are typically not designed to absorb odors. Odor control agents can been incorporated into absorbent articles to absorb, mask or mitigate odors. For example, activated carbon has been included in absorbent articles, such as shoe inserts, to reduce odors. However, activated carbon may be undesirable in certain absorbent article applications because activated carbon can adversely affect the visual properties of the absorbent article, can be difficult to economically and efficiently process into absorbent articles or may adversely increase the stiffness or affect other properties of the absorbent article. Furthermore, activated carbon and other solid odor control agents may not be effective at removing all odors and can become ineffective when wet.

One attempt to include a liquid, specifically glycerin, in an absorbent article was proposed in U.S. Pat. No. 4,880,417 to Yabrov et al. U.S. Pat. No. 4,880,417 describes a pad containing an absorbing layer that further contains glycerin to act as a deodorant to neutralize hydrogen sulfide gas. The glycerin is dissolved in ethyl alcohol. Fibers that are used in the absorbing layer are soaked with the glycerin and ethyl alcohol mixture. The absorbent layer is allowed to dry for several hours until the alcohol evaporates. However, it is not practical or desirable to manufacture “wet” absorbent articles and methods of making articles that require long periods of drying are not economically desirable.

As such, a need currently exists for an improved technique for incorporating one or more liquid, odor-absorbing and/or one or more liquid, odor-mitigating compounds into absorbent articles. The inclusion of a liquid that absorbs or otherwise mitigates odorous compounds in absorbent articles would allow an increase in the number and in the variety of odor control additives or agents that are available for use and incorporation into absorbent articles, especially absorbent personal care articles such as diapers, adult incontinence products, feminine hygiene products and so on.

### SUMMARY OF THE INVENTION

The present invention provides absorbent articles that include a capturing agent and a liquid, wherein the liquid is captured by the capturing agent. In a particularly desirable embodiment, the liquid is a solvent for an odor, an absorbent for an odor or a liquid that neutralizes an odor. For example, an article of the present invention may include a complexing agent or an encapsulating agent and a liquid wherein the liquid is a solvent for an odor that is found in urine, feces, menses and/or perspiration. Desirably, the liquid absorbs one or more odors that are emitted by urine, feces, menses and/or perspiration. The liquid captured by the capturing agent can be complexed in particles of a complexing agent or can be entrapped in particles of an encapsulating agent. Suggested capturing agents that are encapsulating agents include carbohydrates, cyclodextrins, gums, lipids, celluloses, silicates, clays, synthetic polymers, and mixtures thereof. Suggested capturing agents that are complexing agents include, but are not limited to, hydrogel particles, superabsorbent polymer particles, desiccant particles, cyclodextrin particles, synthetic polymer particles and mixtures thereof. Still other suggested encapsulating agents also include nanospheres.

In one particular embodiment, the capturing agent includes synthetic polymer particles wherein the synthetic polymer is a polymer or a copolymer of an acrylate, ethylene oxide or an allyl methacrylate and the synthetic polymer particles comprise from about 0.1 to about 1,000 weight percent of the liquid disposed within or otherwise associated with the particles. In still another embodiment, the capturing agent is a complexing agent that includes superabsorbent polymer particles that have been exposed to an environment of greater than about 90 percent relative humidity. The captured liquid may be or may include water, aqueous mixtures, oils, silicones, alcohols, ethers, esters, ketones, amines, nitrated and chlorinated hydrocarbons and mixtures thereof. Suggested liquids include oil, water and aqueous mixtures. Still more suggested captured liquids include water and aqueous mixtures having a pH of less than 7.0 as well as water and aqueous mixtures having a pH of greater than 7.0. In yet another desirable embodiment, the present invention provides an absorbent article that includes an outercover and an absorbent core wherein particles of the capturing agent are disposed within the outercover or the absorbent core or on a surface of the outercover or the absorbent core. For example, the absorbent article can include an outercover wherein the outercover includes a first layer and a second layer wherein the second layer comprises the capturing agent and from about 0.1 to about 100,000 weight percent of the liquid relative to the weight of the dry capturing agent. The absorbent article may be a personal care product for example pantiliners, diapers, incontinence garments, training pants, swimming pants and so forth.

The present invention also provides a method of making an absorbent product that includes exposing an absorbent product or a component of the absorbent product that includes a capturing agent to water, elevated humidity, an aqueous liquid or another liquid solvent so that the absorbent product or the component of the absorbent product absorbs a quantity of the water, aqueous liquid or other liquid solvent and then packaging the absorbent product In one embodiment, the absorbent product comprises at least about 0.1, at least about 1 and even more than about 10 weight percent of water, aqueous liquid, another liquid solvent or a combination thereof relative to the weight of the absorbent product prior to packaging of the absorbent product. The amount of liquid captured, complexed with or entrapped in the complexing agent may range from greater than about 5 weight percent, greater than about 100 weight percent, greater than about 1000 weight percent and even as much as 100,000 weight percent of liquid relative to the weight of the dry, capturing agent.

The present invention also provides a feminine hygiene product that includes a capturing agent and a liquid, wherein the liquid is captured, complexed with, disposed within or otherwise encapsulated within the capturing agent. Desirably, the liquid is a solvent for an odor, an absorbent for an odor or a liquid that neutralizes an odor that is found in menses and/or urine. The liquid may be complexed with particles of a complexing agent or entrapped in particles of an encapsulating agent.

Other features and aspects of the present invention are discussed in greater detail below.

### BRIEF DESCRIPTION OF THE DRAWINGS

A full and enabling disclosure of the present invention, including the best mode thereof, directed to one of ordinary skill in the art, is set forth more particularly in the specification, which makes reference to the appended figures in which:

FIG. 1 is a top view of an absorbent article, such as a feminine pad.

FIG. 2 is a side view of the absorbent article shown in FIG. 1.

FIG. 3 is an end view of the absorbent article shown in FIG. 1.

FIG. 4 is an enlarged cross-sectional view of the absorbent article shown in FIG. 1 taken along line 4--4.

### DEFINITIONS

As used herein, the term “coform” means a process in which at least one meltblown diehead is arranged near a chute through which other materials are added to the web while it is forming. Such other materials may be pulp, superabsorbent particles, natural polymers (for example, rayon or cotton fibers) and/or synthetic polymers (for example, polypropylene or polyester) fibers, for example, where the fibers may be of staple length. Coform processes are shown in commonly assigned U.S. Pat. Nos. 4,818,464 to Lau and 4,100,324 to Anderson et al. Webs produced by the coform process are generally referred to as coform materials.

As used herein, “complexing agent” means any material or agent that can complex, chelate or otherwise coordinate a liquid so that the liquid is not perceived as dampness by a human's touch, but is available to absorb one or more odors.

As used herein “encapsulating agent” means any material or agent that can physically encapsulate or otherwise entrap a liquid so that the liquid is not perceived as dampness by a human's touch, but is available to absorb one or more odors.

As used herein, “capturing agent” means any material or agent that can complex, chelate or otherwise coordinate a liquid or any material or agent that can encapsulate or otherwise entrap a liquid so that the liquid is not perceived as dampness by a human's touch but is available to absorb one or more odors, and is meant to include both complexing agents and encapsulating agents.

As used herein the term “meltblown fibers” means fibers formed by extruding a molten thermoplastic material through a plurality of fine, usually circular, die capillaries as molten threads or filaments into converging high velocity, usually hot, gas (e.g. air) streams which attenuate the filaments of molten thermoplastic material to reduce their diameter, which may be to microfiber diameter. Thereafter, the meltblown fibers are carried by the high velocity gas stream and are deposited on a collecting surface to form a web of randomly dispersed meltblown fibers. Such a process is disclosed, for example, in U.S. Pat. No. 3,849,241 to Butin et al. Meltblown fibers are microfibers which may be continuous or discontinuous, are generally smaller than 10 microns in average diameter, and are generally tacky when deposited onto a collecting surface.

As used herein, the term “personal care product” means bandages and wound care items, diapers, training pants, swimwear, absorbent underpants, adult incontinence products, and feminine hygiene products such as tampons, panty liners and so forth.

As used herein, a “solvent” includes any substance capable of dissolving another substance, for example liquids capable of dissolving odorous compounds such as trialkylamines. Desirably, the solvent takes into solution and absorbs one or more odorous compounds that may be contained in urine, feces and/or menses and thus reduces the concentration of the odorous compound.

As used herein the term “spunbonded fibers” refers to small diameter fibers which are formed by extruding molten thermoplastic material as filaments from a plurality of fine, usually circular capillaries of a spinneret with the diameter of the extruded filaments then being rapidly reduced as by, for example, in U.S. Pat. No. 4,340,563 to Appel et al., and U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartman, and U.S. Pat. No. 3,542,615 to Dobo et al. Spunbond fibers are generally not tacky when they are deposited onto a collecting surface. Spunbond fibers are generally continuous and have average diameters (from a sample of at least 10) larger than 7 microns, more particularly, between about 10 and 20 microns. The fibers may also have shapes such as those described in U.S. Pat. No. 5,277,976 to Hogle et al., U.S. Pat. Nos. 5,466,410 to Hills and 5,069,970 and 5,057,368 to Largman et al., which describe fibers with unconventional shapes.

### DETAILED DESCRIPTION

Reference now will be made in detail to various embodiments of the invention, one or more examples of which are set forth below. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment, can be used on another embodiment to yield still a further embodiment. Thus, it is intended that the present invention covers such modifications and variations as come within the scope of the appended claims and their equivalents.

In general, the present invention is directed to an absorbent article that includes a capturing agent and a liquid. The capturing agent may be either a complexing agent or an encapsulating agent and includes mixtures thereof. A liquid is complexed with a complexing agent or disposed within or otherwise encapsulated in an encapsulating agent. In a particularly desirable embodiment, the absorbent article of the present invention includes a liquid that is a solvent for an odor, an absorbent for an odor, or a neutralizer for an odor wherein the odor is an odor that may be found in or is associated with urine, feces, perspiration, menses, and so forth. For example, an article of the present invention may include a complexing agent and a complexed liquid or an encapsulating agent and an encapsulated liquid wherein the liquid is a solvent for an odor that is found in urine, feces, menses and/or perspiration. Alternatively, the liquid may be an absorbent for an odor or may mitigate an odor that is found in urine, feces, menses and/or perspiration. The liquid can be complexed in particles of a complexing agent or can be entrapped in particles of an encapsulating agent. Suggested encapsulating agents include, but are not limited to, carbohydrates, cyclodextrins, gums, lipids, celluloses, silicates, clays, synthetic polymers, and mixtures thereof. Suggested complexing agents include, but are not limited to, hydrogel particles, superabsorbent polymer particles, desiccant particles, cyclodextrin particles, synthetic polymer particles and mixtures thereof. Suggested encapsulating agents also include, but are not limited to, nanospheres and solid shell materials capable of encapsulating a liquid while allowing the liquid to be available to absorb one or more odors.

In one particular embodiment, the capturing agent is or includes synthetic polymer particles wherein the synthetic polymer is a polymer or a copolymer of an acrylate, ethylene oxide or an allyl methacrylate. The synthetic polymer particles that include from about 0.1 to about 100,000 weight percent of an odor-absorbing liquid disposed within or otherwise associated with the particles relative to the weight of the synthetic polymer before the synthetic polymer has captured any odor-absorbing liquid, specifically the weight of the synthetic polymer not including the weight of captured odor-absorbing liquid. In still another embodiment, the capturing agent is a complexing agent that includes superabsorbent polymer particles that have been exposed to an environment of greater than about 90 percent relative humidity and have absorbed water. The odor-absorbing liquid may be or may include water, aqueous mixtures, oils, silicones, alcohols, ethers, esters, ketones, amines, nitrated and chlorinated hydrocarbons and mixtures thereof. Suggested odor-absorbing liquids include, but are not limited to, oil, water and aqueous mixtures. Still more suggested odor-absorbing liquids include water and aqueous mixtures having a pH of less than 7.0 as well as water and aqueous mixtures having a pH of greater than 7.0. In yet another desirable embodiment, the present invention provides an absorbent article that includes an outercover and an absorbent core wherein particles of the capturing agent are disposed within the outercover or the absorbent core or on a surface of the outercover or the absorbent core. The capturing agent can be incorporated as particles or as a powder that is dispersed in the absorbent portion of the absorbent article or can be incorporated in one of the film layers or in a nonwoven layer of the absorbent article. For example, an absorbent article can include an outercover wherein the outercover includes a first layer and a second layer wherein the second layer includes a complexing agent or an encapsulating agent and the complexing agent or the encapsulating agent includes from about 0.1 to about 100,000 weight percent of an odor-absorbing liquid relative to the weight of the dry capturing agent. Suggested absorbent articles include, but are not limited to, personal care products: for example panty liners, diapers, incontinence garments, training pants, swimming pants and so forth.

The present invention also provides a method of making an absorbent product that includes exposing an absorbent product or a component of the absorbent product that includes a capturing agent to water, elevated humidity, an aqueous liquid or another liquid solvent so that the capturing agent captures, including complexes or entraps, a quantity of the water, aqueous liquid or other liquid solvent, and then packaging the absorbent product. The product can be packaged in a sealed, relatively air tight container or package to prevent the odor-absorbing liquid form evaporating or absorbing odors before the package is opened and the product is used by a consumer. It is suggested that the absorbent product includes at least about 0.1 weight percent, at least about 1 weight percent and even more than 10 weight percent of water, aqueous liquid, another liquid solvent or a combination thereof relative to the weight of the absorbent product prior to packaging of the absorbent product. The amount of liquid complexed with the complexing agent or entrapped within the encapsulating agent may range from greater than about 5 weight percent, greater than about 100 weight percent, greater than about 1000 percent, greater than about 10,000 percent and even greater than 100,000 weight percent of liquid relative to the weight of the dry complexing agent or dry encapsulating agent, respectively. The weight of the dry capturing agent, either a complexing agent or encapsulating agent, is the weight of the capturing agent before the capturing agent has captured any odor-absorbing liquid, more specifically the weight of the capturing agent not including odor-absorbing liquid.

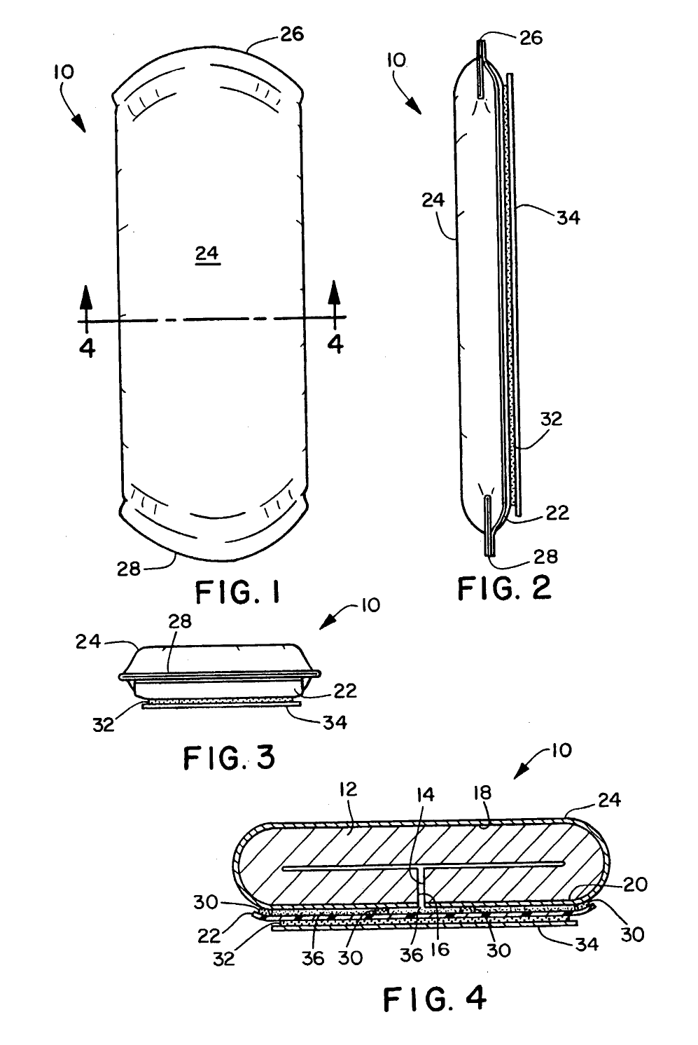

Referring to FIGS. 1-4, an exemplary absorbent article 10 is shown. The absorbent article 10 can be a diaper, a feminine pad, a panty liner, an incontinent garment, an underarm shield, a bed pad, and so forth. For purposes of illustration, the absorbent article 10 is depicted as a feminine pad in FIGS. 1-4. The absorbent article 10 contains an absorbent 12, best shown in FIG. 4. The absorbent 12 can be constructed of and include cellulose, wood fluff, coform, meltblown or carded material, sphagnum moss, or other known absorbent materials. Coform is an air-formed mixture of melt blown and staple fibers, such as wood fluff. The absorbent 12 is shown C-folded having two abutting ends 14 and 16. The absorbent 12 includes a first surface 18, which faces the body of a user, and a second surface 20 aligned approximately opposite to the first surface 18.

The absorbent article 10 also contains a liquid-impermeable baffle 22, which is positioned adjacent to at least the second surface 20 of the absorbent 12. It is possible that the baffle 22 can extend upwards around a portion of the sides of the absorbent 12 and even overlap a portion of the first surface 18 in certain embodiments. The liquid-impermeable baffle 22 can be constructed of, for example, a polypropylene meltblown fabric, polyethylene meltblown fabric or other suitable liquid-impermeable material including, but not limited to polyolefin films. A fluid-permeable cover 24 is positioned adjacent to at least the first surface 18 of the absorbent 12. The cover 24 can extend completely around the absorbent 12, as is shown in FIG. 4. Alternatively, the cover 24 can partially cover the absorbent 12 and be attached directly to the baffle 22 or an intermediate member so that both the cover 24 and the baffle 22 sandwich the absorbent 12 there between. The cover 24, for example, can be a nonwoven, spunbond fabric or a perforated film material, or be made from a material having a netting type texture. The cover 24 can be porous, embossed, perforated or exhibit a smooth surface. The cover 24 is designed to come in contact with the body of the user and present a soft and comfortable feel while exhibiting the ability to allow human exudate in the form of blood, menses, urine or other types of body fluid to readily pass through into the absorbent 12.

When the cover 24 completely surrounds the absorbent 12, as is shown in FIGS. 1-4, opposite ends 26 and 28 of the cover 24 can be bonded together to envelop the absorbent 12. Furthermore, the baffle 22 can be bonded to the outside surface of the cover 24 by a construction adhesive 30. In FIG. 4, four distally spaced longitudinal strips of construction adhesive 30 secure the baffle 22 to the cover 24. It is possible to use only a single strip of adhesive, if one desires. As with most conventional pads, a garment adhesive 32, which can be pressure sensitive, is applied to an outside surface of the baffle 22 and a removable peel strip 34 is attached thereto. The peel strip 34 is designed to be removed just prior to use of the product by a consumer. In use, the consumer removes the peel strip 34 and attaches the product to the inside surface of an undergarment so that the product will remain in position relative to the perineum of the body.

An absorbent article of the present invention includes at least one capturing agent and a liquid for example at least one complexing agent and a liquid that is complexed with the complexing agent or at least one encapsulating agent and a liquid that is disposed within or otherwise encapsulated within the encapsulating agent. For example, a liquid can be encapsulated within a nanosphere. Desirably, the nanospheres are permeable to an odor so that the liquid within the nanospheres can absorb the odor. Generally, the liquid is for absorbing and/or mitigating malodors and odorous compounds. The absorbent article may include one or more complexing agents and a complexed odor-absorbing liquid and one or more encapsulating agent and an encapsulated odor-absorbing liquid or any combination thereof. The capturing agent and the captured liquid may be included in a variety of locations in an absorbent article including but not limited to, the fluid permeable cover 24, the absorbent 12, the surface of the liquid impermeable baffle 22 and so forth, so that the complexed or entrapped liquid can absorb one or more malodors when the absorbent article absorbs body fluids. In one suggested embodiment, particles of a capturing agent including a captured liquid are dispersed as a powder in the fluff that make up the absorbent portion of the absorbent article.

Odors are introduced into an absorbent article 10 when the absorbent 12 absorbs body fluids. Wet absorbent articles are not desirable and it would be advantageous to provide absorbent articles that do not require wetting in order to begin absorbing odors. It has been discovered that an improved absorbent article can be made that includes a liquid solvent that is captured within a capturing agent so that the liquid solvent cannot be felt but is still available to absorb odors. Examples of suggested solvents include, but are not limited to, water, aqueous mixtures, oils, silicones, alcohols, ethers, esters, ketones, amines, nitrated and chlorinated hydrocarbons and mixtures thereof. Particularly suggested solvents include water and aqueous mixtures that have a pH of less than 7.0, water and aqueous mixtures that have a pH of greater than 7.0 and oils. Water absorbs and mitigates water-soluble odorous compounds such as trimethylamine (TMA) and ammonia. Suggested oils include, but are not limited to, mineral oil, linseed oil and so forth. Other suggested solvents include, but are not limited to, silicones such as dimethicones and cyclomethicones. Dimethicones are linear polydimethyl siloxanes with trimethyl siloxy end blocks. Cyclomethicones are cyclic polydimethyl siloxanes. These silicones have limited solubility in water. Other silicones may be water soluble or water dispersible and a can be used as an odor-absorbing liquid of the present invention.

In accordance with the present invention, an absorbent article includes a solvent that is complexed, encapsulated, entrapped or otherwise coordinated within a capturing agent that may be a complexing agent or an encapsulating agent so that the liquid solvent cannot be felt but is present to absorb and/or neutralize one or more odorous compounds. Generally, the capturing agent can be any compound that captures, complexes, encapsulates, entraps, chelates or otherwise coordinates one or more liquid solvents. For example, water can be complexed in superabsorbent particles so that the water cannot be perceived by a consumer but is present to absorb water-soluble odors such as TMA and ammonia. Additionally, the pH of water or an aqueous mixture that is complexed within a complexing agent can be adjusted to provide better or more specific odor control. For example, the pH of the water or aqueous solvent can be selected or adjusted to have a pH of less than 7, e.g. slightly acidic, to improve the ability of the water or the aqueous mixture to absorb alkaline odors, for example amines such as TMA as well as ammonia. Alternatively, the pH of the water or aqueous solvent can be selected or adjusted to have a pH of greater than 7, e.g. slightly alkaline, to improve the ability of the water or aqueous mixture to absorb acidic odors for example fatty acids such as isovaleric acid. Other examples of capturing agents that are encapsulating agents include, but are not limited to, cyclodextrins, gums, lipids, celluloses, clays, and synthetic polymers. Examples of capturing agents that are complexing agents include, but are not limited to, dessicants, hydrogels and mixtures thereof. Suggested synthetic polymer capturing agents include, but are not limited to, polymers and copolymers of ethylene oxide, allyl methacrylates and acrylates. Suggested classes of super absorbent polymers include, but are not limited to, crosspolymers of allyl methacrylate, crosslinked sodium polyacrylates and polymers and copolymers of ethylene oxide.

One suggested class of commercially available allyl methacrylate polymers that can be used as capturing agents, more specifically as complexing agents, includes POLY-PORE® 180CM, 180DM, 280CM and 280DM polymers available from Chemdal Corporation of Arlington Heights, Ill. These POLY-PORE®, polymers are highly adsorptive crosspolymers of allyl methacrylates that have high surface areas and very high intruded volumes. The polymers were developed for controlled release of pharmaceutical and skin care compositions but have other applications. Because POLY-PORE® polymers have large intruded volumes, POLY-PORE® polymers can be used to entrap various liquids that can be used to solubilize, absorb or otherwise mitigate odors. POLY-PORE® polymers may also have improved controlled release properties. POLY-PORE® polymers can be obtained from the supplier with one of various liquids already complexed with the polymer. For example, POLY-PORE® polymers that have been complexed or otherwise loaded with water, mineral oil, benzyl acetate, glycerin, cyclomethicone, dimethicone, and benzophenone are currently commercially available. A suggested class of commercially available polyacrylates that can be used as capturing agents includes DRYTECH® superabsorbents that are available from Dow Chemical Corporation of Midland, Mich. DRYTECH® superabsorbents are granular crosslinked sodium polyacrylates that can be used to absorb aqueous fluid for various personal care applications, such as baby diapers, adult incontinent devices and feminine hygiene products.

In one embodiment, an odor-absorbing liquid is encapsulated within an encapsulating agent having a “solid shell”. The solid shell of the encapsulating agent should be permeable to an odor to allow the odor to be solubilized by the odor-absorbing liquid that is encapsulated within the encapsulating agent. Examples of potential encapsulating agents that have a solid shell include, but are not limited to, carbohydrates, gums, lipids, celluloses, silicates, clays, and synthetic polymers. The encapsulated odor-absorbing liquid would essentially provide an odor-absorbing liquid in a “solid” form that could be incorporated into an absorbent article and not be perceived as dampness by a consumer. A capturing agent having a solid shell that encapsulates an odor-absorbing liquid can be included in an absorbent article in a variety of ways. For example, the capturing agent may be attached to the inside surface of an outercover film of a personal care product or the complexing agent may be included in the absorbent portion of the product, e.g. attached or interspersed in fluff, coform or airlaid fibers in the absorbent portion of a personal care product. Again, more than one odor-absorbing liquid may be included in the capturing agent, and additional complexing agents including additional odor-absorbing liquids may be included in the absorbent article as well as other additives.

The capturing agent or a combination of a plurality of capturing agents, including one or more complexing agents and/or one or more encapsulating agents may combine to complex, encapsulate, entrap, chelate or otherwise coordinate one or more solvents to absorb more than one odor or may include one or more classes of solvents to absorb one or more classes of odors. For example, an absorbent article of the present invention may include a first complexing agent such as an allyl methacrylate polymer that is complexed with a hydrophobic odor-absorbing liquid such as cyclomethicone to absorb hydrophobic odorous compounds such as sulfides, mercaptans, organic fatty acids, ketones aldehydes and so forth and a second complexing agent such as a polyacrylate that is complexed with a second, hydrophilic odor-absorbing liquid such as water to absorb hydrophilic odorous compounds such as ammonia, TMA and so forth. Generally, hydrophilic odor-absorbing liquids are not very effective at absorbing hydrophobic odors and hydrophobic odor-absorbing liquids are not very effective at absorbing hydrophilic odors. Advantageously, an absorbent article that includes more than one complexed or encapsulated odor-absorbing liquid can be made to absorb more than one odorous compound or more than one class of odorous compounds. The multiple odor-absorbing liquids may be included in separate complexing agents or may even be included in the same complexing agent. Other suggested combinations of odor-absorbing liquids include, but are not limited to: at least one acidic liquid (i.e. pH <7) for absorbing alkaline odors and at least one alkaline liquid (i.e. pH >7) for absorbing acidic odors, for example fatty acids associated with perspiration odors; and water or an aqueous solution for absorbing water-soluble odors such as amines and at least one solvent such as an alcohol or an ether for water-insoluble odors such as sulfides. Sulfides can be solubilized and absorbed by certain alcohols and ethers. Cyclodextrins have hydrophobic cavities and could be used to complex an alcohol, a silicone or an ether or a combination thereof to absorb a water-insoluble odor such as dimiethyl disulfide (DMDS). Additionally, one or more solid odor absorbing compounds, such as activated carbon, silica, alumina, titania, clay, magnesia and so forth and combinations thereof may be further included in an absorbent article of the present invention.

The capturing agent may be included in an absorbent article as particles of capturing agent or as a layer of the capturing agent. For example, hydrogel particles can be attached to the inside surface of an outercover of a personal care product, such as a diaper, or a capturing agent can be included as a layer in multilayer outercover, for example, a laminate that includes a polyolefin layer and a hydrogel layer. In one embodiment, dry particles of a capturing agent can be included into the absorbent portion of a personal care product and then the capturing agent is exposed to an odor-absorbing liquid, for example contacted with water or exposed to high humidity, to allow water to complex with the capturing agent. Therefore, one embodiment of the present invention provides for the complexing agent to be included in an absorbent article as particles of the complexing agent. As previously stated, the particles of capturing agent may be included within an outercover or within an absorbent core of the absorbent article or on a surface of the outercover or the absorbent core. In another embodiment, the capturing agent is included as a layer in a multilayer laminate that is a component in an absorbent article. For example, the capturing agent may be a layer of a hydrogel polymer that includes from about 1 to about 100,000 weight percent water, from about 10 to about 10,000 weight percent water, from about 50 to about 5,000 weight percent or from about 100 to about 3,000 weight percent water relative to the weight of the dry hydrogel polymer in a two layer polymer film. The two layer film can be used as an outercover for a diaper.

As stated above, an odor-absorbing additive that is an odor-absorbing liquid that is captured in a capturing agent can be included in a variety of locations in a variety of absorbent products. The capturing agent that includes the odor-absorbing liquid can be inserted or otherwise included into an absorbent article in a variety of ways. For example, the capturing agent and liquid captured therein can be positioned such that it will not be immediately contacted by body fluids discharged by the user. Desirably, the captured liquid is included in the absorbent article in a “dry” state so that the captured liquid is not perceivable, although the complexed liquid will work in the wet condition.

Various bodily fluids contain malodorous chemical compounds including acyclic and cyclic amines, aldehydes, fatty acids, and sulfur-containing compounds such as sulfides. Vaginal secretions and used menstrual pads may contain many malodorous chemical compounds; for example, TMA, pyridine, furaldehyde, isovaleric acid, and methyl mercaptan. And, urine may contain dimethyl disulfide (DMDS). To facilitate laboratory evaluations, a compound with a boiling point significantly above room temperature was chosen as a model to represent various types of chemical compounds. TMA (40% TMA/60% water, boiling point 95° C.) and DMDS (boiling point 109° C.) were chosen as representatives of the types of odoriferous chemical compounds to which an absorbent article might be exposed.

The particular malodorous compounds, which will be emitted by various absorbent articles, will vary depending upon the person wearing the absorbent article and the type of fluid absorbed; i.e., urine, menstrual fluid, perspiration, milk, etc. For feminine pads, the length of time that the article is worn, the quantity of fluid which is absorbed, and the exposure of the pad to different bodily fluids will determine which odors can be emitted by the absorbent article.

### EXAMPLES 1-6

Adsorptive polymers of allyl methacrylate, specifically POLY-PORE® 180DM, 180CM, 280DM and 280CM polymers obtained from Chemdal Corporation of Arlington Heights, Ill. were tested for odor absorbing capacity. The 180 and 280 allyl methacrylate cross polymers were obtained from Chemdal Corporation loaded with about 10 grams of dimethicone (DM) or cyclomethicone (CM) solvent per gram of allyl methacrylate polymer as reported in their literature. Samples of the loaded polymers were tested for their ability to absorb odor, both TMA and DMDS. TMA is a water-soluble odorous compound and DMDS is a water-insoluble odorous compound. The results of the tests are presented below.

### Odor Reduction Test Method

To evaluate the odor removing properties of the above exemplary embodiments of the present invention, Examples 1-6, samples of material were exposed to two odorous compounds, either TMA or dimethyl disulfide (DMDS). Trimethylamine is a water soluble odorous compound and dimethyl disulfide is a water insoluble odorous compound. Each sample of odor absorbing/removing material was tested using the following procedure.

The procedure involves determining how much of a known amount of odor is removed by a known amount of sample. Both sample and odor are sealed in a 20-cubic centimeter headspace vial and kept at 37° C. for 10 minutes prior to the analysis. Samples were typically tested in duplicate. The data are reported as “mg odor removed per gram of sample.”

The ability of a sample to remove certain odiferous compounds was determined using a test known as “Headspace Gas Chromatography.” DMDS obtained form Aldrich (99.0+% purity) was used to determine the ability of the samples to remove malodorous sulfur compounds. Trimethylamine (TMA) obtained from Aldrich as a 40 percent TMA and 60 percent water solution was used to determine the ability of a sample to remove malodorous amine compounds.

The headspace gas chromatography testing was conducted on an Agilent Technologies 5890, Series II gas chromatograph with an Agilent Technology 7694 headspace sampler (Agilent Technologies, Waldbronn, Germany). Helium was used as the carrier gas (injection port pressure: 12.7 psig; headspace vial pressure: 15.8 psig; supply line pressure is at 60 psig). A DB-624 column was used for the TMA and dimethyl disulfide (DMDS) analyses that had a length of 30 meters and an internal diameter of 0.25 millimeters. Such a column is available from J&W Scientific, Inc. of Folsom, Calif.

The operating parameters used for the headspace gas chromatography are summarized in Table 1 below.

TABLE 1
Operating Parameters for the Headspace Gas Chromatography Device
Headspace Parameters

| | | |
|---|---|---|
| Zone Temps, ° C. | Oven | 37 |
| | Loop | 42 |
| | TR. Line | 47 |
| Event Time, minutes | GC Cycle time | 10.0 |
| | Vial eq. Time | 10.0 |
| | Pressuriz. Time | 0.20 |
| | Loop fill time | 0.20 |
| | Loop eq. Time | 0.15 |
| | Inject time | 0.30 |
| Vial Parameters | First vial | 1 |
| | Last vial | 1 |
| | Shake | [off] |

The test procedure involved placing 0.0100 gram of the particulate sample in a 20-cubic centimeter headspace vial. Using a syringe, an aliquot of the odoriferous agent (TMA or DMDS) was also placed in the vial. The vial was then sealed with a cap and a septum and placed in the headspace gas chromatography oven at 37° C. After ten minutes, a hollow needle was inserted through the septum and into the vial. A 1-cubic centimeter sample of the headspace (air inside the vial) was then injected into the gas chromatograph.

Initially a control vial with only the aliquot of odoriferous agent (TMA or DMDS) was tested to define 0% odoriferous agent adsorption. To calculate the amount of headspace odoriferous agent removed by the sample, the peak area for the odoriferous agent from the vial with the sample was compared to the peak area from the odoriferous agent control vial. Testing was done with 3.8 milligrams of DMDS (3.6 microliters), 1.95 milligrams of TMA (5.0 microliters of 40% TMA in a water solution), and about 0.0100 gram of the particulate sample. Each sample was tested in duplicate.

The results are set forth below in Tables 2 and 3 in terms of milligrams (mg) odor adsorbed per gram (g) of the sample, i.e., the “Relative Adsorption Efficiency.” The results represent the average of two measurements per samples.

TABLE 2
Test data for POLY-PORE ® samples

| Example no. | Sample Description | DMDS ¹ | TMA ² |
|---|---|---|---|
| 1 | POLY-PORE ® E200 ^{a} | 56.0 ± 3.7 | 6.6 ± 4.4 |
| 2 | POLY-PORE ® 180CM | 106.5 ± 5.0 | 2.7 ± 2.9 |
| 3 | POLY-PORE ® 180DM | 86.0 ± 1.1 | 5.5 ± 2.6 |
| 4 | POLY-PORE ® L200 ^{b} | 73.3 ± 1.3 | 13.6 ± 8.0 |
| 5 | POLY-PORE ® 280CM | 104.3 ± 5.2 | 11.8 ± 0.5 |
| 6 | POLY-PORE ® 280DM | 85.2 ± 4.0 | 11.4 ± 14.3 |

¹ mg of DMDS removed per gram of sample
² mg of TMA removed per gram of sample
^{a} hydrophilic and lipophilic; the starting material for POLY-PORE ® 180CM and 180DM (i.e. unloaded or not loaded with “CM” or “DM”)
^{b} lipophilic; the starting material for POLY-PORE ® 280CM and 280DM (i.e. unloaded or not loaded with “CM” or “DM”)

The test data in Table 2 confirm that POLY-PORE® polymers loaded with cyclomethicone (CM) or dimethicone (DM), Examples 2, 3, 5 and 6, are more efficient at removing water-insoluble malodorous compounds such as dimethyl disulfide (DMDS). By comparison, unloaded POLY-PORE® polymers, i.e. polymers that have not been loaded with a water-insoluble solvent, such as cyclomethicone or dimethicone (Examples 1 and 4), are not as effective at removing dimethyl disulfide. However, the POLY-PORE® L200 polymer of Example 4 is an oleophilic and hydrophobic polymer that is lipophilic and may be used to absorb, entrap or complex an oil to mitigate odors that are soluble in oils. The POLY-PORE® E200 polymer of Example 1 is both hydrophilic and lipophilic, and therefore should be able to absorb, entrap or complex an oil or an aqueous solvent to mitigate odors that are soluble in oils or aqueous solvents. It is believed that capturing agents that include at least one solvent will absorb and remove malodorous compounds that are found in urine, feces and/or menses that are soluble in that particular solvent, and capturing agents that include multiple solvents should absorb and remove odors that are soluble in the multiple solvents that have been included in the capturing agent(s). Note in Table 2 that POLY-PORE® multifunctional polymers loaded with CM or DM are not very effective at removing water soluble odors such as TMA as expected since CM and DM are not good solvents for water-soluble odors.

### EXAMPLE 7 AND COMPARATIVE EXAMPLE 7A

Superabsorbent particles that include complexed water as a solvent were produced by placing DRYTECH 2035M superabsorbent particles obtained from Dow Chemical Company of Midland, Mich. in an environmental chamber for about two and one half days with conditions measured at 100 percent relative humidity and 37° C. Headspace gas chromatography data for a water-insoluble odor, DMDS, were collected on the same day that the superabsorbent particles complexed with water were removed from the environmental chamber, Example 7. The amount of complexed water was measured at 0.5 gram of water per gram of superabsorbent particles. Headspace gas chromatography data for a water soluble odor, TMA, were also collected one day after the particles had been removed from the chamber. The amount of complexed water was also measured two days after the superabsorbent particles with complexed water had been removed from the chamber. The amount of complexed water had dropped to 0.2 gram of water per gram of superabsorbent particles after two days, apparently due to exposure to lower ambient humidity. Table 3 presents the headspace gas chromatography data for the superabsorbent particle samples that were complexed with water as an odor-absorbing solvent, Example 7, and for “dry” superabsorbent particles as obtained from the supplier, Example 7A.

TABLE 3
Headspace Gas Chromatography Data for
Superabsorbent Particles with and without Complexed Water

| Example no. | Sample Description | DMDS ¹ | TMA ² |
|---|---|---|---|
| 7A | DRYTECH 2035M SAP | 2.3 ± 2.8 | 55.5 ± 2.1 |
| | (dry) without complexed water | | |
| 7 | DRYTECH 2035M SAP | 4.8 ± 0.5 | 74.6 ± 9.4 |
| | complexed with water | | |

¹ mg of DMDS removed per gram of sample
² mg of TMA removed per gram of sample

The test data presented in Table 3 confirm that DRYTECH 2035M superabsorbent particles that include a quantity of complexed water are effective at absorbing and removing a water-soluble odor such as TMA. The test data also illustrate that DRYTECH 2035M superabsorbent particles that include a complexed water are more effective at absorbing and removing a water-soluble odor, such as TMA, than “dry” DRYTECH 2035M superabsorbent particles, i.e. superabsorbent particles that do not include complexed water. It is believed that superabsorbent particles that include complexed water will act as a solvent to other water-soluble malodorous compounds, such as ammonia, that may be found in urine, feces and/or menses.

While the invention has been described in detail with respect to the specific embodiments thereof, it will be appreciated that those skilled in the art, upon attaining an understanding of the foregoing, may readily conceive of alterations to, variations of, and equivalents to these embodiments. Accordingly, the scope of the present invention should be assessed as that of the appended claims and any equivalents thereto.

What is claimed is:

1. An absorbent article comprising a capturing agent and a liquid, wherein the liquid is captured by the captured agent.

2. The absorbent article of claim 1 wherein the liquid is a solvent for an odor, an absorbent for an odor or a liquid that neutralizes an odor.

3. The absorbent article of claim 1 wherein the capturing agent is a complexing agent and the liquid is complexed with the complexing agent.

4. The absorbent article of claim 1 wherein the capturing agent is an encapsulating agent and the liquid is encapsulated within the complexing agent.

5. The absorbent article of claim 1 wherein the capturing agent is selected from the group consisting of carbohydrates, cyclodextrins, gums, lipids, celluloses, silicates, clays, synthetic polymers, and mixtures thereof.

6. The absorbent article of claim 1 wherein the capturing agent comprises particles selected from the group consisting of hydrogel particles, superabsorbent polymer particles, desiccant particles, cyclodextrin particles, synthetic polymer particles and mixtures thereof.

7. The absorbent article of claim 1 wherein the capturing agent comprises synthetic polymer particles wherein the synthetic polymer is a polymer or a copolymer of an acrylate, ethylene oxide or an allyl methacrylate and the synthetic polymer particles comprise from about 0.1 to about 100,000 weight percent of the weight of liquid disposed within or otherwise associated with the particles.

8. The absorbent article of claim 1 wherein the capturing agent comprises superabsorbent polymer particles that have been exposed to an environment of greater than about 90 percent relative humidity.

9. The absorbent article of claim 1 wherein the liquid is selected from the group consisting of water, aqueous mixtures, oils, silicones, alcohols, ethers, esters, ketones, amines, nitrated and chlorinated hydrocarbons and mixtures thereof.

10. The absorbent article of claim 1 wherein the liquid is water or an aqueous mixture.

11. The absorbent article of claim 1 wherein the liquid is water or an aqueous mixture and has a pH of less than 7.0.

12. The absorbent article of claim 1 wherein the liquid is water or an aqueous mixture and has a pH of greater than 7.0.

13. The absorbent article of claim 1 wherein the liquid is an oil.

14. The absorbent article of claim 1 wherein the liquid is a silicone.

15. The absorbent article of claim 6 wherein the absorbent article comprises an outercover and an absorbent core and particles of the capturing agent are disposed within the outercover or the absorbent core or on a surface of the outercover or the absorbent core.

16. The absorbent article of claim 1 wherein the absorbent article comprises an outercover and the outercover comprises a first layer and a second layer wherein the second layer comprises the capturing agent and from about 0.1 to about 100 weight percent of the liquid relative to the weight of the capturing agent.

17. The absorbent article of claim 1 wherein the absorbent article is a personal care product selected from the group consisting of pantiliners, diapers, incontinence garments, training pants, and swimming pants.

18. An absorbent article comprising a complexing agent and a liquid, wherein the liquid is complexed by the complexing agent.

19. The absorbent article of claim 18 wherein the liquid is a solvent for an odor, an absorbent for an odor or a liquid that neutralizes an odor.

20. The absorbent article of claim 18 wherein the complexing agent comprises particles selected from the group consisting of hydrogel particles, superabsorbent polymer particles, desiccant particles, cyclodextrin particles, synthetic polymer particles and mixtures thereof.

21. The absorbent article of claim 18 wherein the liquid is selected from the group consisting of water, aqueous mixtures, oils, silicones, alcohols, ethers, esters, ketones, amines, nitrated and chlorinated hydrocarbons and mixtures thereof.

22. The absorbent article of claim 18 wherein the liquid is water or an aqueous mixture.

23. The absorbent article of claim 18 wherein the liquid is water or an aqueous mixture and has a pH of less than 7.0.

24. The absorbent article of claim 18 wherein the liquid is water or an aqueous mixture and has a pH of greater than 7.0.

25. The absorbent article of claim 18 wherein the liquid is an oil.

26. The absorbent article of claim 18 wherein the liquid is a silicone.

27. An absorbent article comprising a encapsulating agent and a liquid, wherein the liquid is encapsulated by the encapsulating agent.

28. The absorbent article of claim 27 wherein the liquid is a solvent for an odor, an absorbent for an odor or a liquid that neutralizes an odor.

29. The absorbent article of claim 27 wherein the encapsulating agent comprises particles selected from the group consisting of carbohydrates, cyclodextrins, gums, lipids, celluloses, silicates, clays, synthetic polymers, and mixtures thereof.

30. The absorbent article of claim 27 wherein the liquid is selected from the group consisting of water, aqueous mixtures, oils, silicones, alcohols, ethers, esters, ketones, amines, nitrated and chlorinated hydrocarbons and mixtures thereof.

31. The absorbent article of claim 27 wherein the liquid is water or an aqueous mixture.

32. The absorbent article of claim 27 wherein the liquid is water or an aqueous mixture and has a pH of less than 7.0.

33. The absorbent article of claim 27 wherein the liquid is water or an aqueous mixture and has a pH of greater than 7.0.

34. The absorbent article of claim 27 wherein the liquid is an oil.

35. The absorbent article of claim 27 wherein the liquid is a silicone

36. A method of making an absorbent product comprising a capturing agent, the method comprising exposing an absorbent product or a component of the absorbent product comprising the capturing agent to water, elevated humidity, an aqueous liquid or another liquid solvent so that the absorbent product or the component of the absorbent product absorbs a quantity of the water, aqueous liquid or other liquid solvent and then packaging the absorbent product.

37. The method of claim 36 wherein the absorbent product comprises at least about 0.1 weight percent of water, aqueous liquid, another liquid solvent or a combination thereof relative to the weight of the absorbent product prior to packaging of the absorbent product.

38. A feminine hygiene product comprising a capturing agent and a liquid, wherein the liquid is captured by the capturing agent.

39. The feminine hygiene product of claim 38 wherein the liquid is a solvent for an odor, an absorbent for an odor or a liquid that neutralizes an odor.

40. The feminine hygiene product of claim 38 wherein the capturing agent is a complexing agent and the liquid is complexed in particles of the complexing agent.

41. The feminine hygiene product of claim 38 wherein the capturing agent is an encapsualting agent and the liquid is encapsulated in particles of the encapsulating agent.

42. An absorbent article comprising a capturing agent and a liquid, wherein the liquid encapsulated within the capturing agent or complexed with the capturing agent and the liquid is a solvent for an odor.

43. The absorbent article of claim 42 wherein the capturing agent comprises nanopheres.
